(19) United States
(12) Patent Application Publication
(10) Pub. No.: US 2019/0298699 A1
(43) Pub. Date: Oct. 3, 2019

(54) COMPOSITIONS OF THIOREDOXIN MIMETICS AND ITS USE

(71) Applicant: Illustris Pharmaceuticals, Inc., Irvine, CA (US)

(72) Inventor: Jacob M. WAUGH, Corona del Mar, CA (US)

(21) Appl. No.: 16/366,730

(22) Filed: Mar. 27, 2019

Related U.S. Application Data
(60) Provisional application No. 62/649,308, filed on Mar. 28, 2018.

(51) Int. Cl. A61K 31/425; A61K 9/00; A61P 29/00; A61P 17/06; A61P 17/10

(52) U.S. Cl. CPC A61K 31/425; A61K 9/0014; A61P 17/10; A61P 17/06; A61P 29/00

(57) ABSTRACT

Disclosed herein are methods and compositions that can be used to treat inflammatory skin conditions. In one embodiment, a method of treating an inflammatory skin condition comprises administering topically a composition comprising an effective amount of a thioredoxin mimetic.

### CROSS REFERENCE

This application claims priority to U.S. Provisional Application No. 62/649,308, filed on Mar. 28, 2018, titled “Compositions of Thioredoxin Mimetics and Its Use” and is incorporated herein by reference.

### SUMMARY

Disclosed herein the methods and compositions comprising thioredoxin mimetics for treating various skin disorders and aging. In one embodiment, a method of treating an inflammatory skin condition in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof.

In another embodiment, a method of treating photoaging in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof.

In a further embodiment, a method of treating UV-induced skin damage in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof.

Also disclosed herein are compositions comprising a thioredoxin mimetic selected from the group consisting of a compound of formula I, a compound of formula II, a compound of formula III, a compound of formula IV, a compound of formula V, a thioredoxin mimetic peptide, and a combination thereof. In some embodiments, the compositions are topical compositions.

### DETAILED DESCRIPTION

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

All percentages, parts and ratios are based upon the total weight of the topical compositions and all measurements made are at about 25° C., unless otherwise specified.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

Free radicals are formed in tissues in multiple ways, for example, as a result of exposure to sunlight. Free radicals initiate chain reactions that produce hydroxyls, superoxides and other radicals, which can damage DNA and other macromolecules. Other processes that generate free radicals are respiration and phagocytosis. To control the damage done by these free radicals, mammals have a number of antioxidant systems. Some of them are obtained from the diet, like vitamin C, E, carotenoids, and polyphenols; and others are produced endogenously (glutathione, thioredoxin).

Insufficient or unsuitable antioxidants may lead to oxidative stress and the consequent oxidation of membrane lipids, damage to DNA, etc. Damage by free radicals formed in response to ultraviolet (UV) light is now believed to be a major cause of skin aging, with the term “photo-aging” being used to describe the multitude of effects of sunlight on the skin, including wrinkles, changes in the matrix proteins elastin and collagen, “age” spots (also called sun spots) and an increase in the incidence of skin cancer. Topical application of thioredoxin can help alleviate skin problems caused by sunlight or other types of radiation producing free radicals, such as inflammation.

Thioredoxins are small proteins with a redox active disulfide bridge present in the characteristic active site sequence: -Trp-Cys-Gly-Pro-Cys- (tryptophane-cysteine-glycine-proline-cysteine). Thioredoxins have a molecular mass of approximately 12,000. Animals, plants, and most microorganisms possess a system in which thioredoxin is reduced by NADPH and the enzyme NADP-thioredoxin reductase. Reduced thioredoxins have been found to serve as electron donors in a variety of cellular redox reactions. The defense against oxidative damage by UV-generated free radicals has been found to be mediated by thioredoxin/thioredoxin reductase system. Disclosed herein the methods and compositions comprising thioredoxin mimetics that may be used topically for treating various skin disorders and aging.

Various embodiments of the invention are directed to topical compositions comprising an effective amount of one or more thioredoxin mimetics, and pharmaceutically acceptable excipients, salts, or solvates thereof.

In some embodiments, the thioredoxin mimetic is 1-(2-acetamido-3-mercaptopropanoyl)-N-(1-amino-3-mercapto-l-oxopropan-2-yl)pyrrolidine-2-carboxamide, represented by formula (I):

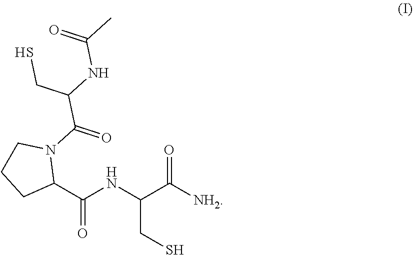 [SMILES: CC(=O)NC(CS)C(=O)N1CCCC1C(=O)NC(CS)C(N)=O]

or a pharmaceutically acceptable salt or solvate thereof.

In some embodiments, the thioredoxin mimetic is 2-(1-(2-amino-3-mercaptopropanoyl) pyrrolidine-2-carboxamido)-3-mercaptopropanoic acid, represented by formula (II):

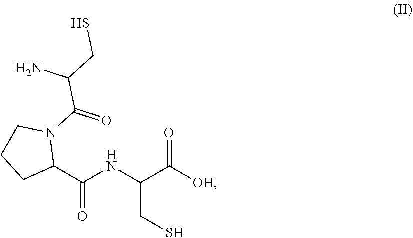 [SMILES: NC(CS)C(=O)N1CCCC1C(=O)NC(CS)C(=O)O]

or a pharmaceutically acceptable salt or solvate thereof.

In some embodiments, the thioredoxin mimetic is represented by formula III as follows:

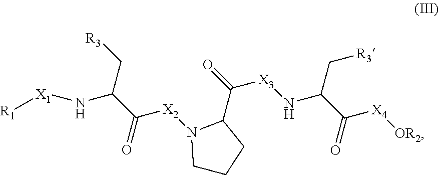

or a pharmaceutically acceptable salt or solvate thereof, wherein

X₁ is absent or an amino acid residue forming a peptide bond with the —NH— group adjacent thereto;

X₂ is absent or an amino acid residue forming peptide bonds with the carbonyl group and nitrogen atom adjacent thereto;

X₃ is absent or an amino acid residue forming peptide bonds with the carbonyl and —NH— groups adjacent thereto;

X₄ is absent or an amino acid residue forming a peptide bond with the carbonyl group adj acent thereto;

R₁ is H, —CO(C₁-C₈)alkyl, —CO(C1-C₈)alkylene-NH((C₁-C₈)alkyl, —CO(C₁-C₈)alkylene-N((C₁-C₈)alkyl)₂, or —CO(C₁-C₈)alkylene-N⁺((C₁-C₈)alkyl)₃, covalently linked to the N atom of the amino moiety of X₁, when present;

R₂ is H or (C₁-C₈)alkyl, covalently linked to the carbonyl moiety of X₄, when present;

R₃ and R₃′ each independently is —SH, —S—CO(C₁-C₈)alkyl, —S—CO(C₁-C₈)alkylene— N((C₁-C₈)alkyl)₂, or —S—CO(C₁-C₈)alkylene-N⁺((C₁-C₈)alkyl)₃; or R₃ and R₃′ are both —S— and together form a disulfide bond; or one of R₃ and R₃′ is —S— forming a disulfide bond with one of R₃ and R₃′ of another identical or different compound of the formula III, and another of R₃ and R₃′ is —SH, —S—CO(C₁-C₈)alkyl, —S—CO(C₁-C₈)alkylene-N((C₁-C₈)alkyl)₂, or —S—CO(C₁-C₈)alkylene-N⁺((C₁-C₈)alkyl)₃; or R₃ and R₃′ are both —S—, wherein R₃ forms a disulfide bond with one of R₃ and R₃′ of another identical or different compound of the formula III, and R₃′ forms a disulfide bond with either another of R₃ and R₃′ of said another compound of the formula III, or with one of R₃ and R₃′ of a further identical or different compound of the formula III, provided that at least one of R₁ and R₂ is not H, or at least one of R₃ and R₃′ is not —SH, but excluding the compounds wherein R₃ and R₃′ are each —SH, R₁ is —CO(Ci-C₈)alkyl, R₂ is H, and Xi, X₂, X₃ and X₄ are absent.

In some embodiments, the thioredoxin mimetic is represented by the formula IV:

A-Y1-Cys-Y2-Cys-Y3-B (IV)

wherein, Cys is a cysteine residue,
A is the first hydrophobic or non-charged moiety;

B is the second hydrophobic or non-charged moiety; Y1, Y2 and Y3 are each individually one or more amino acid residues in the range of 0-30 residues, with the provision that Y1, Y2 and Y3 collectively provide for at least two amino acid residues in the peptide. In some embodiments, A is selected from the group consisting of N-acetyl, tert-butyl, isopropyl, n-butyl and n-pentyl. In some embodiments, B is an amide or an ester.

In some embodiments, the thioredoxin mimetic is of formula V:

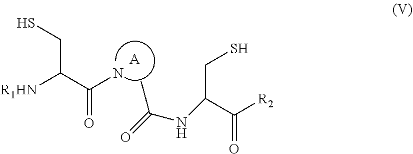

wherein R₁ is H, —CO(C₁-C₈)alkyl, —COO(C₁-C₈)alkyl or —CONH(C₁-C₈)alkyl;

R₂ is OH or N(R₃R₄);

R₃ and R₄ each independently is H, (C₁-C₈)alkyl, (C₃-C₁₀) cycloalkyl, 4-12-membered heterocyclyl, or (C₆-C₁₄)aryl;

A is a 3-6 membered ring optionally containing one or more additional heteroatoms selected from sulfur, oxygen or nitrogen, wherein said nitrogen atom may be substituted by (C₁-C₈)alkyl, and each one of the carbon atoms in said ring may be substituted by oxo, halogen, (C₁-C₈)alkyl, (C₆-C₁₄)aryl, 4-12-membered heterocyclyl, NO₂, N(R₅R₆), —OR₅, —SR₅, —SO₂R₅, or —COR₇, or two adjacent carbon atoms in said ring form a 3-6 membered saturated, partially saturated, or aromatic carbocyclic or heterocyclic ring;

R₅ and R₆ each independently is H, or (C₁-C₈)alkyl; and

R₇ is OH, NH₂, or —O(C₁-C₈)alkyl, but excluding the compounds wherein R1 is H or —COCH₃, R₂ is OH or NH₂, and A is pyrrolidin-1,2-diyl.

In some embodiments, the thioredoxin mimetic is a peptide selected from N-acetyl-Cys-Pro-Cys-amide, N-acetyl-Cys-Met-Lys-Cys-amide, N-acetyl-Cys-Gly-Pro-Cys-amide, N-acetyl-Cys-amide, or N-acetyl-Cys-Y2-Cys-amide, wherein Y2 is any amino acid.

In some embodiments, the compositions may include one or more thioredoxin mimetics from about 0.1 wt. % to about 25 wt. %, about 0.1 wt. % to about 20 wt. %, about 0.1 wt. % to about 15 wt. %, about 0.1 wt. % to about 10 wt. %, about 0.1 wt. % to about 8 wt. %, about 0.1 wt. % to about 5 wt. %, about 0.1 wt. % to about 4 wt. %, about 0.1 wt. % to about 3 wt. %, about 0.1 wt. % to about 3 wt. %, or about 0.1 wt. % to about 1 wt. % of the total composition. Specific examples include about 0.1 wt. %, about 0.5 wt. %, about 1 wt. %, about 2 wt. %, about 5 wt. %, about 10 wt. %, about 25 wt. %, and ranges between any two of these values. The weight percentages disclosed herein may be weight-to-weight or weight -to-volume percentages with respect to the total amount of the composition.

In some embodiments, the thioredoxin mimetics disclosed herein may be present from about 1 microgram to about 10 milligrams per mL of the composition, about 1 microgram to about 5 milligrams per mL of the composition, about 1 microgram to about 1 milligram per mL of the composition, or about 1 microgram to about 100 micrograms per mL of the compositions.

The compositions of various embodiments may include any of the thioredoxin mimetics identified above or combinations thereof in an effective amount. For example, such compositions for topical administration may be in the form of, but not limited to, solutions, powders, fluid emulsions, fluid suspensions, solid, semi-solids, pastes, creams, gels, jellies, hydrogels, mousse, ointment, foams, lotions, or aerosols.

The compositions disclosed herein may further contain a sensation modifying agent selected from the group of a cooling agent, a warming agent, a relaxing or soothing agent, a stimulating or refreshing agent, and mixtures thereof

In some embodiments, the cooling agent is selected from but not limited to menthol; an isomer of menthol, a menthol derivative; 4-Methyl-3-(1-pyrrolidinyl)-2[5H]-furanone; WS-23, Icilin, Icilin Unilever Analog, 5-methyl-4-(1-pyrrolidinyl)-3-[2H]-furanone; 4,5 -dimethyl-3 -(1-pyrrolidinyl)-2[5H]-furanone; isopulegol, 3-(1-menthoxy)propane-1,2-diol, 3 -(1-menthoxy)-2-methylpropane-1,2-diol, p-menthane-2, 3-diol, p-menthane-3, 8-diol, 6-isopropyl-9-methyl- 1,4-dioxas-piro[4, 5]decane-2-methanol, menthyl succinate and its alkaline earth metal salts, trimethylcyclohexanol, N-ethyl-2isopropyl-5 -methylcyclohexanecarb-oxamide, Japanese mint (Mentha arvensis) oil, peppermint oil, menthone, menthone glycerol ketal, menthyl lactate, 3 -(1-menthoxy)ethan- 1-ol, 3 -(1-menthoxy)propan- 1-ol, 3 -(1-menthoxy)butan-1-ol, 1-menthylacetic acid N-ethylamide, 1-menthyl-4-hydroxypentanoate, 1-menthyl-3 -hydroxybutyrate, N,2,3 -trimethyl-2-(1-methylethyl)-butanamide and spearmint oil.

In some embodiments, the warming agent is selected from but not limited to polyhydric alcohols, capsaicin, capsicum powder, a capsicum tincture, capsicum extract, capsaicin, hamamalis, homocapsaicin, homodihydrocapsaicin, nonanoyl vanillyl amide, nonanoic acid vanillyl ether, vanillyl alcohol alkyl ether derivatives, such as vanillyl ethyl ether, vanillyl butyl ether, vanillyl pentyl ether, and vanillyl hexyl ether, isovanillyl alcohol alkyl ethers, ethylvanillyl alcohol alkyl ethers, veratryl alcohol derivatives, substituted benzyl alcohol derivatives, substituted benzyl alcohol alkyl ethers, vanillin propylene glycol acetal, ethylvanillin propylene glycol acetal, ginger extract, ginger oil, gingeol and gingeron.

In some embodiments, the relaxing or soothing agent is selected from but not limited to herb extracts, selected from the group consisting of aloe vera, alpha bisabolol, D-panthenol, allantoin, hamamelis, chamomile, yarrow; calendula, comfrey, witch hazel and other astringents, sea weed, and oat extracts; oils, selected from the group consisting of: almond oil, avocado oil, and comfrey; and essential oils, selected from the group consisting of: cardamone, eucalyptus, mentha piperita (peppermint), hyssop, and rosemary; waxy or unctuous substances selected from the group consisting of: lanolin or vaselline jelly, minerals, selected from the group consisting of: zinc oxide, calamine and selenium; vitamins, selected from the group consisting of: tocopheryl acetate (vitamin E), and pharmaceutical agents selected from the group consisting of: analgesics, anesthetics, anti-inflammatory agents, and anti-histamines, and muscle relaxants; menthol, camphor, eugenol, eucalyptol, safrol, methyl salicylate, menthyl lactate, menthyl ethoxyacetate, menthone glycerinacetal, 3-1-menthoxypropane-1,2-diol, ethyl 1-menthyl carbonate, (1S, 3S,4R)-p-menth-8-en-3 -ol, menthyl pyrrolidone carboxylate, N-substituted-p-menthane-3-carboxamides hamamelis extract and ginger oil.

In some embodiments, the stimulating or refreshing agent is selected from but not limited to an alcohol, L-menthol, camphor, menthe oil, capsicum extract, capsaicin, benzyl nicotinate, salicylate, glycol salicylate, acetyl choline, serotonin, histamine, a prostaglandin, a neurotransmitter; a CNS stimulant, caffeine and quinine

In some embodiments, the compositions described herein may further include one or more cosmetic or pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

In some embodiments, the compositions described herein may be formulated as a liquid. Liquid dosage forms for topical administration may include diluents such as, for example, alcohols, glycols, oils, water, and the like. Such compositions may also include wetting agents or emulsifiers. In some embodiments, the compositions of embodiments may be formulated as oil-in-water or water-in-oil emulsion. A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e., carrier) include hydrocarbons (petrolatum, beeswax, etc.) vegetable oils, fatty alcohols (cholesterol, lanolin, wool alcohol, stearyl alcohol, etc.) or silicones. Insoluble solids such as starch, zinc oxide, calcium carbonate, or talc can also be used in ointments and creams. Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

Emollient or lubricating vehicles that help hydrate the skin can also be used. Examples of suitable bases or vehicles for preparing hydrating compositions for use with a subject skin are petrolatum, petrolatum plus volatile silicones, lanolin, cold cream (USP), and hydrophilic ointment (USP).

In particular embodiments, the compositions described above can be formulated as aerosols in which the composition is dissolved in a propellant such as dichlorodifluoromethane, trichlorofluoromethane, dichlorotetrafluoroethane, carbon dioxide, or other suitable gas, and a co-solvent such ethanol, acetone, hexadecyl alcohol, and the like and combinations thereof.

In certain embodiments, the compositions of various embodiments may be formulated for improving enhance the texture, appearance, color, sensation, or hydration of the skin and may additionally include additives such as vitamins, cosmetic peptides, oil control agents, and other skin care agents.

Vitamins include, for example, vitamin D, vitamin K, vitamin B (including niacinamide, nicotinic acid, C1-18 nicotinic acid esters, and nicotinyl alcohol; B6 compounds, such as pyroxidine; and B5 compounds, such as panthenol, or “pro-B5”), vitamin A (including retinoids such as retinyl propionate, carotenoids, and other compounds), vitamin E (including tocopherol sorbate, tocopherol acetate, other esters of tocopherol), vitamin C (including ascorbyl esters of fatty acids, and ascorbic acid derivatives, for example, ascorbyl glucoside, magnesium ascorbyl phosphate, sodium ascorbyl phosphate, and ascorbyl sorbate), and all natural and/or synthetic analogs thereof, and combinations thereof. In various embodiments, the compositions may include about 0.0001 wt. % to about 50 wt. %, about 0.001 wt. % to about 10 wt. %, about 0.01 wt. % to about 5 wt. %, or about 0.1 wt. % to about 1 wt. %, or any individual concentration or range of each vitamin contained in the composition.

Cosmetic peptides include di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (MATRIXYL®) palmitoyl-glycine-glutamine-proline-arginine (RIGIN®), these three being available from Sederma, France, and Cu-histidine-glycine-glycine (Cu-HGG, also known as IAMIN®), and naturally occurring and synthesized derivatives thereof, and combinations thereof. In various embodiments, the compositions may include about 1×10−7 wt. % to about 20 wt. %, about 1×10−6 wt. % to about 10 wt. %, and about 1×10−5 wt. % to about 5 wt. %, or any individual concentration or range of each peptide contained in the composition.

Oil control agents include compounds useful for regulating the production of skin oil, or sebum, and for improving the appearance of oily skin. Examples of oil control agents include, for example, salicylic acid, dehydroacetic acid, benzoyl peroxide, vitamin B3 (for example, niacinamide), and the like, their isomers, esters, salts and derivatives, and mixtures thereof. The compositions of such embodiments may include about 0.0001 wt. % to about 15 wt. %, about 0.01 wt. % to about 10 wt. %, about 0.1 wt. % to about 5 wt. %, and about 0.2 wt. % to about 2 wt. %, or any individual concentration or range of each oil control agent contained in the composition.

Other skin care agents include retinol, steroids, sunblock, salicylate, minocycline, antifungals, peptides, antibodies, lidocaine, and the like and combinations thereof. In some embodiments, other skin care agents include N-acyl amino acid compounds including, for example, N-acyl phenylalanine, N-acyl tyrosine, and the like, their isomers, including their D and L isomers, salts, derivatives, and mixtures thereof. An example of a suitable N-acyl amino acid is N-undecylenoyl-L-phenylalanine is commercially available under the tradename SEPIWHITE®. Other skin active agents include, but are not limited to, Lavandox, Thallasine 2, Argireline NP, Gatuline In-Tense and Gatuline Expression, Myoxinol LS 9736, Syn-ake, and Instensyl®, Sesaflash™, N- acetyl D-glucosamine, panthenol (for example, DL panthenol available from Alps Pharmaceutical Inc.), tocopheryl nicotinate, benzoyl peroxide, 3-hydroxy benzoic acid, flavonoids (for example, flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (for example, retinyl propionate), phytic acid, N-acetyl-L-cysteine, lipoic acid, tocopherol and its esters (for example, tocopheryl acetate: DL-a-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′- trichlorocarbanilide, octopirox, lidocaine hydrochloride, clotrimazole, miconazole, ketoconazole, neomycin sulfate, theophylline, and mixtures thereof

In some embodiments, the compositions can also include skin lightening agents, such as ascorbic acid compounds, vitamin B3 compounds, azelaic acid, butyl hydroxyanisole, gallic acid and its derivatives, glycyrrhizinic acid, hydroquinone, kojic acid, arbutin, mulberry extract, and mixtures thereof Use of combinations of skin lightening agents is believed to be advantageous in that they may provide skin lightening benefit through different mechanisms.

In some embodiments, the compositions include sunblock agents, such as but not limited to para-aminobenzoic acid (PABA), PABA esters (glyceryl PABA, amyldimethyl PABA and octyldimethyl PABA), benzophenones (oxybenzone and sulisobenzone), cinnamates (octylmethoxy cinnamate and cinoxate), salicylates (homomethyl salicylate) anthranilates, TiO2, avobenzone, bemotrizinol, bisoctrizole, 3-(4-methylbenzylidene)-camphor, cinoxate, diethylamino hydroxybenzoyl hexyl benzoate, dioxybenzone, drometrizole trisiloxane, ecamsule, ethylhexyl triazone, homosalate, menthyl anthranilate, octocrylene, octyl salicylate, iscotrizinol, isopentenyl-4-methoxycinnamate, octyl- dimethyl-p-aminobenzoic acid, octyl-methoxycinnamate, oxybenzone, polysilicone-15, trolamine salicylate, and ZnO. In some embodiments, any thioredoxin mimetics disclosed herein can be combined with any of the sunblock agents disclosed herein or known in the art.

In some embodiments, the compositions can be in the form of hydrogels. Hydrogels are typically prepared by cross-linking various monomers and/or polymers to provide a three-dimensional polymer network. Non-limiting examples of polymers include, polyoxyethylene-polypropylene block copolymers, ionic poly saccharides, such as chitosan or sodium alginate, cellulose, and biodegradable polymers, such as poly-lactides (PLA) and poly-glycolides (PGA), butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG).

In some embodiments, the compositions disclosed herein can be in the form of transdermal patches. The transdermal patches can be in any conventional form such as, for example, a strip, a gauze, a film, and the like. Patch material may be nonwoven or woven (e.g., gauze dressing). Layers may also be laminated during processing. It may be nonocclusive or occlusive, but the latter is preferred for backing layers. The patch is preferably hermetically sealed for storage (e.g., foil packaging). The patch can be held onto the skin and components of the patch can be held together using various adhesives. For example, the transdermal patch can be in the form of a bandage type device, or it may be packaged in a small metal or plastic “cup”, which is strapped onto the appropriate site using an adhesive, tape, or an outer fabric or leather strap, similar to that worn as part of a watch. The entire patch may be disposable or may be refillable.

### Methods of Using the Compositions Described Herein

In some embodiments, the compositions disclosed herein can be used to treat inflammatory skin conditions. In some embodiments, a method of treating an inflammatory skin condition in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof. Non-limiting examples of inflammatory skin conditions include dermatitis, acne vulgaris, psoriasis, rosacea, warts, keratosis, hyperhidrosis, cutaneous scars, skin irritation, inflammatory acne, papule, pustule, nodule, cyst, and others.

In some embodiments, the thioredoxin mimetic compositions disclosed herein can be used to treat dermatitis. Dermatitis (also called eczema) is generic inflammation of the skin. Specific types of dermatitis include atopic, contact, nummular, and photo-induced. Contact dermatitis is an inflammatory condition of the skin either of irritant exposure to the skin without specific adaptive immunologic pathogenesis or of allergic sensitization and subsequent exposure of the skin to the sensitizing allergen with specific adaptive immunologic pathogenesis. Atopic dermatitis is a genetically determined disease that is part of the broader disease complex of atopy that includes asthma, hay fever, and atopic dermatitis.

In some embodiments, the thioredoxin mimetic compositions disclosed herein can be used to treat acne vulgaris. Acne vulgaris, a progressively inflammatory disorder of the pilosebaceous follicular unit especially of the face and upper chest and back is a very common disease of both males and females after initiation of puberty. The methods disclosed herein can be used to treat common acne, comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne or acne medicamentosa.

In some embodiments, the thioredoxin mimetic compositions disclosed herein can be used to treat rosacea. Rosacea is a chronic condition characterized by facial erythema and sometimes pimples. Rosacea typically begins as redness on the central face across the cheeks, nose, or forehead, but can also less commonly affect the neck, chest, ears, and scalp. There are 3 subtypes of rosacea that affect the skin: erythematotelangiectatic rosacea, papulopustular rosacea, and phymatous rosacea.

Other non-limiting inflammatory skin conditions that may be treated with the thioredoxin mimetic compositions disclosed herein are Human Papilloma Virus induced lesions, for example, warts, common warts, palmoplantar warts, flat warts, epidermodysplasia verruciformis related warts, anogenital warts, condyloma accuminatum; Herpesvirus related lesions including those induced by HHV-1 (HSV-1), HHV-2 (HSV-2), HHV-3 (varicella-zoster virus) e.g. chicken pox, Herpes zoster, shingles; Poxvirus induced lesions e.g. molluscum contagiosum, orf; callus, cutaneous horns, corns, acrochordons, fibroepithelial polyps, prurigo nodularis, actinic keratoses, squamous cell carcinoma, squamous cell carcinoma in situ, keratoacanthoma, basal cell carcinoma, cutaneous lymphomas and benign lymphocytic infiltrates & hyperplasias of the skin, clear cell acanthoma, large cell acanthoma, epidermolytic acanthoma, porokeratosis, hyperkeratosis, lichenoid keratosis, acanthosis, acanthosis nigricans, confluent and reticulated papillomatosis, nevi, including e.g., dermal nevi, epidermal nevi, compound nevi, ILVEN (inflammatory linear verrucous epidermal nevi), nevus sebaceous, nevus comedonicus, and the like; acne, e.g., comedonal acne, inflammatory acne, papular acne, pustular acne, cystic acne, cysts, e.g., epidermoid cysts, milia, trichilemmal cysts, follicular cysts, proliferating cysts, dermoid cysts, pilonidal cysts, apocrine cysts, eccrine cysts, sebaceous cysts, mucous cysts, myxoid cysts, ganglion cysts, synovial cysts, vellus hair cysts, steatocystoma, hidrocystoma; adnexal neoplasms e.g., trichofolliculoma, fibrofolliculoma, perifollicular fibroma, trichodiscoma, nevus sebaceous, chondroid syringoma, trichoepithelioma, trichoblastoma, desmoplastic trichoepithelioma, pilomatricoma, pilomatrical carcinoma, tricholemmoma, trichelemmal carcinoma, tumor of the follicular infundibulum, tricoadenoma, proliferating pilar tumor, sebaceous hyperplasia, sebaceous adenoma, sebaceous epithelioma, sebaceous carcinoma, syringoma, poroma, hidradenoma, apocrine hidradenoma, spiradenoma, cylindroma, eccrine nevus (eccrine hamartoma), papillary adenoma, papillary adenocarcinoma; Benign melanocytic neoplasms e.g. ephilides, café-au-lait macules, Becker's melanosis, lentigines, solar lentigines, lentigo simplex, mucosal melanocytic lesions, Mongolian spots, Nevus of Ota, blue nevus, common acquired melanocytic nevi (nevocellular nevus, “moles”), congenital nevi, nevus spilus, recurrent nevi; vascular and perivascular neoplasms and reactive hyperplasias e.g., hemangiomas, cherry angiomas, hobnail hemangiomas (targeted hemosiderotic hemangiomas), tufted angiomas, hemangioendotheliomas, angiolymphoid hyperplasia with eosinophilia (ALHE), Glomus tumors (glomangiomas), hemangiopericytomas; cutaneous neural and neuroendocrine neoplasms e.g., neuromas, Schwannomas, neurofibromas, nerve sheath tumor, nerve sheath myxoma, neurothekeoma, granular cell tumor; fibrotic and fibrohistiocytic proliferations e.g., acrochordons, fibroepithelial polyps, fibromas, fibrous papules, angiofibromas, pearly penile papules, periungual fibromas, dermatofibromas, fibrokeratomas, sclerotic or pleomorphic fibromas, connective tissue nevi; cutaneous scars, hyperplasias, keloids, rosacea, cutaneous fungal, dermatophyte & mold infections, onychomycosis, hyperpigmentation, rhytides, psoriasis, malignant melanoma, seborrheic keratosis, seborrheic keratosis variants including e.g. dermatosis papulosis nigra, inverted follicular keratosis/keratoma warty dyskeratosis/warty dyskeratoma, acrokeratosis verruciformis, stucco keratosis, hyperhidrosis, pachyonychia congenita, and combinations thereof

In some embodiments, the thioredoxin mimetic compositions disclosed herein can be used to treat photoaging. Photoaging, or premature aging, is a process in which the skin changes in appearance as a result of repeated exposure to sunlight. Typically, photoaging occurs in areas of habitual exposure, such as the scalp, face, ears, neck, chest, forearms, and hands. The changes associated with photoaging include elastosis, atrophy, wrinkling, vascular changes (diffuse erythema, ecchymoses, and telangiectasias), pigmentary changes (lentigines, freckles, and areas of hypo- and hyper-pigmentation), and the development of seborrheic keratosis, actinic keratosis, comedones, and cysts. In some embodiments, a method of treating photoaging in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof

In some embodiments, the thioredoxin mimetic compositions disclosed herein can be used to treat conditions of UV-induced skin damage. In some embodiments, a method of treating UV-induced skin damage in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof. Non-limiting examples of UV-induced skin damage include, skin lesions, wrinkles, hyperpigmentation, dysplasias such as actinic keratosis, and malignant skin tumors such as squamous cell or basal cell carcinoma.

In some embodiments, the thioredoxin mimetic compositions disclosed herein can be used to treat various conditions, such as but not limited to chronic pain, post-operative pain, urinary incontinence, neurological disorders (Alzheimer's, dementia, Parkinson's, restless leg syndrome, depression, neuropathic pain, schizophrenia, sleep disturbance, cognitive disorder), angina, coronary heart disease, COPD, nausea, motion sickness, contraceptive, hormonal therapy, arthritis, osteoarthritis, rheumatoid arthritis, inflammatory bowel disease, addiction, ADHD, anti-inflammatory conditions, skin disorders, breast cancer, erectile dysfunction, vitamin deficiency, calcium deficiency, diabetes, diabetic neuropathy, diabetic foot, post-menopause symptoms, hot flashes, hormone replacement therapy, migraine, herpes infection, gingival inflammation, renal failure, Tinnitus, tennis elbow, tendonitis, lipolysis, carpal tunnel syndrome, hypogonadism, avascular necrosis, induction of labor, peripheral neuropathic pain, spinal cord injury, oral mucositis, and hypertension. In some embodiments, the thioredoxin mimetic compositions may be used to treat skin burns caused due to, for example UV radiation, radiotherapy, chemical exposure, environmental exposures, and others.

In some embodiments, the thioredoxin mimetic compositions disclosed herein may be used to treat conditions related to hair, hair shaft, hair follicles, hair bulbs, oil glands, and components thereof, including, for example, hair loss, dandruff, seborrheic dermatitis, alopecia areata, hair disease, ringworm, tinea capitis, folliculitis, pattern hair loss, telogen effluvium, cradle cap, trichotillomania, traction alopecia, trichorrhexis nodosa, folliculitis decalvans, head lice infestation, frontal fibrosing alopecia, non-scarring hair loss, pityriasis amiantacea, dissecting cellulitis of the scalp, acne keloidalis nuchae, monilethrix, pediculosis, alopecia totalis, pseudopelade of Brocq, bubble hair deformity, hair casts, hypertrichosis, ingrown hair, monilethrix, premature greying of hair, pattern hair loss, trichorrhexis invaginata, and the like.

In some embodiments, the thioredoxin mimetic compositions disclosed herein may be used to treat conditions related to nails, such as Beau's lines, onycholysis, onychomycosis, onychoschizia, paronychia, onychocryptosis, mycosis, yellow nail syndrome, onychorrhexis, koilonychias, subungual hematoma, leukonychia, psoriatic onychodystrophy, stipple nails, onychogryphosis, and the like.

In some embodiments, the thioredoxin mimetic compositions disclosed herein may be used to treat conditions related to eye, such as amblyopia, blepharitis, chalazion, conjunctivitis, corneal abrasion, dry eye, diabetic retinopathy, glaucoma, keratitis, hordeolum, uveitis, sty, and the like.

In some embodiments, the thioredoxin mimetic compositions disclosed herein may be used to treat conditions related to buccal or oral cavity, such as oral cancer, thrush, ulcers, gingivitis, sores, leukoplakia, smoker's palate, oral candidosis, bacterial and viral infections, and the like.

In some embodiments, the thioredoxin mimetic compositions disclosed herein may be used to treat conditions related to nasal cavity, such as rhinitis, nasal polyps, sinus infection, upper respiratory tract infections, and the like.

In some embodiments, the thioredoxin mimetic compositions disclosed herein may be used to treat vaginal diseases, such as vaginitis, vaginal discharge, gonorrhea, bacterial vaginosis, sexually transmitted diseases, atrophic vaginitis, yeast infection, genital wart, vaginal cancer, and the like.

In some embodiments, the thioredoxin mimetic compositions disclosed herein may be used to treat conditions related to anus, such as hemorrhoids, anal cancer, pruritus ani, anal fistula, and the like.

In some embodiments, the thioredoxin mimetic compositions disclosed herein may be used to treat conditions related to tongue, such as median rhomboid glossitis, atrophic glossitis, fissured tongue, geographic tongue, oral hairy leukoplakia, lichen planus, linea alba, squamous cell carcinoma, papilloma, macroglossia, and the like.

In some embodiments, the thioredoxin mimetic compositions disclosed herein may be used to treat sinus conditions, such as sinusitis, rhinosinusitis, acute sinusitis, subacute sinusitis, subacute rhinosinusitis, chronic sinusitis, chronic rhinosinusitis, acute exacerbation of chronic rhinosinusitis, fungal sinus disease, sinusitis with polyps, sinus tumors, and the like.

In some embodiments, the thioredoxin mimetic compositions disclosed herein may be used to treat respiratory tract conditions, such as lung cancer, interstitial lung disease, pulmonary embolism, chronic obstructive pulmonary disease, pneumonia, pneumothorax, pulmonary hypertension, pleural effusion, non-small cell lung cancer, asthma, pulmonary fibrosis, obstructive lung disease, respiratory disease, sarcoidosis, bronchitis, tuberculosis, idiopathic pulmonary fibrosis, cystic fibrosis, traction bronchiectasis, pneumonitis, respiratory failure, bronchiolitis, hypersensitivity pneumonitis, restrictive lung disease, usual interstitial pneumonia, lung infection, acute, respiratory distress syndrome, pleurisy, pneumoconiosis, coalworker's pneumoconiosis, hypoxemia, dermatomyositis, burning chest pain, pneumocystis pneumonia, and the like.

In some embodiments, the thioredoxin mimetic compositions disclosed herein may be used to treat conditions associated with lacrimal ducts, such as nasolacrimal duct obstruction, partial lacrimal duct obstruction, total lacrimal duct obstruction, dacryostenosis, dacryoadenitis, dacryocystitis, congenital dacryocystitis, ocular infection, and the like.

In some embodiments, the thioredoxin mimetic compositions disclosed herein may be used to treat conditions associated with inner ear, such as inner ear infection, Meniere's disease, vertigo, autoimmune inner ear disease, noise-induced hearing loss, acoustic neuroma, benign paroxysmal positional vertigo, drug-induced ototoxicity, herpes zoster oticus, purulent labyrinthitis, vestibular neuronitis, and the like.

In some embodiments, the thioredoxin mimetic compositions disclosed herein may be used to treat bladder conditions, such as bladder cancer, urinary tract infection, cystocele, interstitial cystitis, overactive bladder, urinary, incontinence, urinary bladder disease, urinary retention, benign prostatic hyperplasia, neurogenic bladder dysfunction, vesicoureteral reflux, stress incontinence, gastrointestinal disease, nocturnal enuresis, and the like.

In some embodiments, the thioredoxin mimetic compositions disclosed herein may be used to treat kidney conditions, such as acute kidney failure or injury, pyelonephritis, chronic kidney disease, polycystic kidney disease, kidney disease, glomerulonephritis, kidney pain or stone, lupus erythematosus, nephrotic syndrome, nephritis, diabetic nephropathy, IgA nephropathy, autosomal dominant polycystic, kidney disease, cystic kidney disease, renal cyst, alport syndrome, renal tubular acidosis, goodpasture syndrome, medullary sponge kidney, and the like.

In some embodiments, the thioredoxin mimetic compositions disclosed herein may be used to treat urinary tract conditions, such as hydronephrosis, bacteriuria, hematuria, bacterial, fungal, and yeast infections, and the like.

In some embodiments, the thioredoxin mimetic compositions disclosed herein may be used to improve skin texture. In some embodiments, improving skin texture is selected from improving luminosity, quality and combinations thereof.

In some embodiments, the thioredoxin mimetic compositions disclosed herein may be used to improve the execution of motor tasks and/or functional performance in a subject. In some embodiments, the improving the execution of motor tasks and/or functional performance in a subject in need thereof includes improvement in fine motor skills of a subject, due to, for example, extreme or excessive temperatures (warm or cold).

In some embodiments, the thioredoxin mimetic compositions disclosed herein may be used to treat indications selected from Reynaud's syndrome, claudication, peripheral vascular disease, and combinations thereof.

In some embodiments, the thioredoxin mimetic compositions disclosed herein may be used to treat conditions caused by advanced glycation end products (AGEs). Dietary advanced glycation end products (dAGEs) are known to contribute to increased oxidant stress and inflammation, and are linked to onset of several diseases, such as diabetes, heart disease, kidney failure, Alzheimer's disease, and premature aging.

In some embodiments, topical administration includes methods for delivering thioredoxin mimetic to a surface tissue of a subject. A “surface tissue” includes any surface tissue such as, but not limited to, skin, mucosa, eyes, ears, inside the nose, inside the mouth, lips, urethral openings, vagina, anus, tongue, frenulum of tongue, hair, teeth, bone, lacrimal glands, sinus mucosa, respiratory tract, gums, and the like. In some embodiments, the surface tissue is a skin surface or a mucosal surface. In some embodiments, mucosal surface can be eye. In some embodiments, mucosal surface can be oral cavity or vaginal cavity.

In some embodiments, administration of the composition is by topical application, transdermal, percutaneous, or microneedle injection. Administration can also be, for example, intravenous, intraperitoneal, subdermal, subcutaneous, intradermal, transcutaneous, intramuscular, oral, intra-joint, parenteral, intranasal, or by inhalation. Suitable sites of administration thus include, but are not limited to, the skin, bronchium, gastrointestinal tract, eye, buccal cavity, and ear. In some embodiments, the compositions disclosed herein can be administered to any solid tissue via a needle. Such tissues include liver tissue, lung tissue, tissues of the GI tract, muscle tissue, nervous tissue, bone, buccal tissue, and the like.

In some embodiments, the compositions include thioredoxin protein. The thioredoxin protein can be derived from any source, including mammals, and can be produced by genetic engineering techniques. In some embodiments, the compositions comprising thioredoxin protein can be used to treat inflammatory skin conditions, or photoaging, or UV-damaged skin conditions, which are disclosed herein. In some embodiments, the compositions can include thioredoxin protein and a thioredoxin mimetic disclosed herein.

The methods of such embodiments may include a variety of additional steps including, for example, cleaning the surface tissue at the site of applying and the like. In some embodiments, the composition can be topically applied to the surface tissue 1, 2, 3, 4, or more times each day, and applying can be carried out for a period of at least 1 month, 2 months, 3 months, 4 months, 6 months, 8 months or 12 months.

In some embodiments, the composition can be topically applied to the surface tissue 1, 2, 3, 4, or more times each day, and applying can be carried out for a period of at least 1 month, 2 months, 3 months, 4 months, 6 months, 8 months or 12 months. In some embodiments, the composition may be administered once, as needed, once daily, twice daily, three times a day, once a week, twice a week, every other week, every other day, or the like. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In other embodiments, the methods may be used in conjunction with various cosmetic therapies for improving, for example, skin thickness, elasticity, resiliency, smoothness, tone, texture, brightness, clarity, contour, firmness, tautness, suppleness, discoloration, skin lesions, and the like and combinations thereof. The methods of further embodiments can be used for enhancing the color or strength of, for example, hair or teeth. In still other embodiments, the methods of the invention can be used for administering thioredoxin mimetics for treating numerous systemic conditions in which transdermal delivery of the thioredoxin mimetics is preferred, for example, chronic pain relief, cancer, motion sickness, chronic illnesses, and the like and combinations thereof.

The methods disclosed herein may include a variety of additional steps including, for example, cleaning the surface tissue at the site of applying and the like. For example, prior to administering the composition the tissue surface is ablated by electromagnetic radiation, laser, dermal abrasion, chemical peel, ultrasound, heating, cooling, or by a needle.

Abrasion of the outer layer or epidermis of the skin (dermal abrasion) is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin.

Microdermabrasion refers generally to a procedure in which the surface of the skin is removed due to mechanical rubbing by a handpiece emitting a stream of sand or grit. For example, a handpiece can be used to direct an air flow containing tiny crystals of aluminum oxide, sodium chloride, or sodium bicarbonate. The momentum of the grit tends to wear away two to three cell layers of the skin with each pass of the handpiece. Alternatively, new “crystal-free” microdermabrasion techniques utilize a diamond-tipped handpiece without a stream of grit.

In some embodiments, prior to administering the composition the tissue surface is ablated with electromagnetic radiation, for instance using a so-called fractional laser treatment. By way of example, such methods employ electromagnetic radiation (EMR) having one or more wavelengths of between approximately 1,850 to 100,000 nanometers and with pulse widths of between approximately 1 femtosecond (1×10⁻¹⁵ s) to 10 milliseconds (10×10⁻³ s) with fluence in the range of from approximately 1 J/cm² to 300 J/cm². In other examples, the tissue is ablated with electromagnetic radiation having one or more wavelengths of between approximately 2,200 to 5,000 nanometers. In still other examples, the tissue is ablated with electromagnetic radiation having one or more wavelengths of between approximately 190 to 320 nanometers with fluence in the range of from 1 J/cm² to 300 J/cm². Optionally, conditions selected for ablating portions of the tissue minimize the coagulation zone of tissue damage, for instance by keeping the coagulation zone to a relatively small diameter surrounding the ablated void.

Electromagnetic radiation (EMR), particularly in the form of laser light or other optical radiation, has been used in a variety of cosmetic and medical applications, including uses in dermatology, dentistry, ophthalmology, gynecology, otorhinolaryngology and internal medicine. For most dermatological applications, EMR treatment can be performed with a device that delivers the EMR to the surface of the targeted tissue(s). EMR treatment is typically designed to (a) deliver one or more particular wavelengths (or a particular continuous range of wavelengths) of energy to a tissue to induce a particular chemical reaction, (b) deliver energy to a tissue to cause an increase in temperature, or (c) deliver energy to a tissue to damage or destroy cellular or extracellular structures, such as for skin remodeling. Examples of devices that have been used to treat the skin during cosmetic procedures such as skin rejuvenation include the Palomar® LuxIR, the Palomar® 1540, 1440 and 2940 Fractional Handpieces, the Reliant Fraxel® SR Laser and similar devices by Lumenis, Alma Lasers, Sciton and many others.

In some embodiments, the composition is administered by microneedle injection. Microneedle is a hollow needle having an exposed height of between about 0 and 1 mm and a total length of between about 0.3 mm to about 2.5 mm. Preferably, the microneedle is a hollow needle having a length of less than about 2.5 mm. Most preferably, the microneedle is a hollow needle having a length of less than about 1.7 mm. The compositions are delivered into the skin to a depth of at least about 0.3 mm and no more than about 2.5 mm by the microneedle.

In some embodiments, the methods and compositions disclosed herein can be used in combination with photodynamic therapy. Photodynamic therapy is a minimally invasive two-step medical procedure that uses photoactivatable drugs called photosensitizers to treat a range of diseases. First, a photosensitizer is administered and, once it has permeated the target tissue, the photosensitizer is then activated by exposure to a dose of electromagnetic (usually light) radiation at a particular wavelength. The compositions disclosed herein may contain a photosensitizer.

In some embodiments, any suitable photosensitizing agent or mixture of agents may be used herein. Generally, these will absorb radiation in the range of from about 380 nm to about 900 nm. As used herein, “photosensitizer” or “photosensitizing agent” preferably means a chemical compound which, when contacted by radiation of a certain wavelength, forms singlet oxygen or thermal energy. Non-limiting examples of photosensitizers include aminolevulinic acid esters, porphyrins, porphyrin derivatives, bacteriochlorins, isobacteriochlorins, phthalocyanine, naphthalocyanines, pyropheophorbides, sapphyrins, texaphyrins, tetrahydrochlorins, purpurins, porphycenes, phenothiaziniums, and metal complexes such as, but not limited to, tin, aluminum, zinc, lutetium, and tin ethyl etiopurpurin (SnET2), and combinations thereof

In some embodiments, the thioredoxin mimetic compositions disclosed herein can be delivered intranasally. In some embodiments, the compositions can be an aerosol formulation. In this context, the term “aerosol formulation” may refer to an aqueous composition, a dry powder composition, or a propellant-based composition. An aerosol formulation may be delivered to a subject in different ways, for example nasally or perorally, or by inhalation.

In some embodiments, the thioredoxin mimetic composition may be an aqueous solution formulation adapted for pulmonary delivery via a nebulizer, including jet, vibrating mesh, and static mesh or orifice nebulizers.

In some embodiments, the thioredoxin mimetic composition may be a dry powder comprising micronized particles, the particles having diameters from 0.1 to 10 microns and a mean diameter of between about 0.5 to 4.5 microns, about 1 to 4 microns, about 1 to 3.5 microns, about 1.5 to 3.5 microns, or about 2 to 3 microns. The dry powder formulation is suitable for use in either a dry powder inhaler device (DPI) or a pressurized metered dose inhaler (pMDI).

In some embodiments, the thioredoxin mimetic compositions disclosed herein may be in the form of propellant-based formulation which may also be referred to generically herein as “a pMDI formulation”. A pMDI formulation is suitable for delivery by a device such as a pressurized metered dose inhaler (pMDI). In some embodiments, the compositions further comprise a propellant. Suitable propellants are known in the art and include, for example, halogen-substituted hydrocarbons, for example fluorine-substituted methanes, ethanes, propanes, butanes, cyclopropanes or cyclobutanes, particularly 1,1,1,2-tetrafluoroethane (HFA134a) and 1,1,1,2,3,3,3-heptafluoropropane (HFA227), or mixtures thereof

### EXAMPLES

### Example 1

A hydrogel containing 0.5 wt. % thioredoxin mimetic N-acetyl-Cys-Pro-Cys-amide will be applied topically to a wound caused by UV radiation. The oxidative damage by UV-generated free radicals will be inhibited by thioredoxin mimetic. As a result, the wound heals faster and achieve normal function with minimal scarring.

### Example 2

Compositions will be prepared containing 0.1 wt. % thioredoxin mimetic N-acetyl-Cys-Met-Lys-Cys-amide. Control formulations containing saline alone will also be prepared. The compositions will be applied to subject's skin surface affected with rosacea. Subjects treated with compositions containing thioredoxin mimetic are expected to display less inflammatory symptoms and faster healing, relative to comparable controls.

What is claimed is:

1. A method of treating an inflammatory skin condition in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof

2. The method of claim 1, wherein the inflammatory skin condition is selected from the group consisting of dermatitis, psoriasis, rosacea, warts, keratosis, hyperhidrosis, cutaneous scars, skin irritation, inflammatory acne, papule, pustule, nodule, cyst, and combinations thereof

3. The method of claim 1, wherein the thioredoxin mimetic is present from about 0. 1 wt. % to about 5 wt. % of the composition.

4. The method of claim 1, wherein the thioredoxin mimetic peptide is selected from the group consisting of N-acetyl-Cys-Pro-Cys-amide, N-acetyl-Cys-Met-Lys-Cys-amide, N-acetyl-Cys-Gly-Pro-Cys-amide, N-acetyl-Cys-Y2-Cys-amide, wherein Y2 is any amino acid, N-acetyl-Cys-amide, and any combination thereof.

5. The method of claim 1, wherein the topical administration is the only step taken for treating the inflammatory skin condition.

6. A method of treating photoaging in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof.

7. The method of claim 6, wherein the thioredoxin mimetic is present from about 0.1 wt. % to about 5 wt. % of the composition.

8. The method of claim 6, wherein the thioredoxin mimetic peptide is selected from the group consisting of N-acetyl-Cys-Pro-Cys-amide, N-acetyl-Cys-Met-Lys-Cys-amide, N-acetyl-Cys-Gly-Pro-Cys-amide, N-acetyl-Cys-Y2-Cys-amide, wherein Y2 is any amino acid, N-acetyl-Cys-amide, and any combination thereof.

9. The method of claim 6, wherein the topical administration is the only step taken for treating the photoaging.

10. A method of treating UV-induced skin damage in a subject comprises topically administering to the subject in need thereof a composition comprising an effective amount of a thioredoxin mimetic selected from the group consisting of a compound of formula I, a compound of formula II, a compound of formula III, a compound of formula IV, a compound of formula V, a thioredoxin mimetic peptide, and a combination thereof.

11. The method of claim 10, wherein the UV-induced skin damage is selected from skin lesions, wrinkles, hyperpigmentation, actinic keratosis, malignant skin tumors, and combination thereof.

12. The method of claim 10, wherein the thioredoxin mimetic is present from about 0. 1 wt. % to about 5 wt. % of the composition.

13. The method of claim 10, wherein the thioredoxin mimetic peptide is selected from the group consisting of N-acetyl-Cys-Pro-Cys-amide, N-acetyl-Cys-Met-Lys-Cys-amide, N-acetyl-Cys-Gly-Pro-Cys-amide, N-acetyl-Cys-Y2-Cys-amide, wherein Y2 is any amino acid, N-acetyl-Cys-amide, and any combination thereof.

14. The method of claim 10, wherein the topical administration is the only step taken for treating the UV-induced skin damage.

15. A composition comprising a thioredoxin mimetic selected from the group consisting of a compound of formula I, a compound of formula II, a compound of formula III, a compound of formula IV, a compound of formula V, a thioredoxin mimetic peptide, and a combination thereof.

16. The composition of claim 15, wherein the thioredoxin mimetic peptide is selected from the group consisting of N-acetyl-Cys-Pro-Cys-amide, N-acetyl-Cys-Met-Lys-Cys-amide, N-acetyl-Cys-Gly-Pro-Cys-amide, N-acetyl-Cys-Y2-Cys-amide, wherein Y2 is any amino acid, N-acetyl-Cys-amide, and any combination thereof.

17. The composition of claim 15, wherein the composition is a topical composition.
